(19) United States
(12) Patent Application Publication
(10) Pub. No.: US 2014/0080751 A1
(43) Pub. Date: Mar. 20, 2014

(54) INTERNAL OLEFINIC SULFONATE COMPOSITION AND CLEANSING COMPOSITION CONTAINING THE SAME

(71) Applicant: KAO CORPORATION, Tokyo (JP)

(72) Inventors: Yohei YOSHIKAWA, Wakayama-shi (JP); Yoshinori MITSUDA, Wakayama-shi (JP); Hiroshi HORI, Wakayama-shi (JP); Yoshifumi NISHIMOTO, Wakayama-shi (JP); Yasuhiro DOI, Kainan-shi (JP)

(73) Assignee: KAO CORPORATION, Tokyo (JP)

(21) Appl. No.: 13/727,238

(22) Filed: Dec. 26, 2012

(30) Foreign Application Priority Data
Sep. 20, 2012 (JP) 2012-207570

(51) Int. Cl. A61K 8/46; A61Q 19/10; A61Q 5/02

(52) U.S. Cl. CPC A61K 8/466; A61Q 5/02; A61Q 19/10
USPC 510/498; 510/536

(57) ABSTRACT

Provided is an internal olefin sulfonate composition which is capable of exerting good foamability at the same time with foam quality, foam dissipation property, and less irritation to the skin at high levels, and a cleansing composition containing the same.

The internal olefin sulfonate composition of the present invention comprises (A) an internal olefin sulfonate having 16 carbon atoms and (B) an internal olefin sulfonate having 18 carbon atoms, wherein a mass content ratio (A/B) of component (A) to component (B) is from 75/25 to 90/10.

### FIELD OF THE INVENTION

The present invention relates to an internal olefin sulfonate composition useful as a base for a cleansing agent, and to a cleansing composition containing the same.

### BACKGROUND OF THE INVENTION

Anionic surfactants, particularly, alkyl sulfates and alkyl polyoxyalkylene sulfates, are excellent in detergency and foaming power, and thus are widely used as cleansing ingredients for domestic or industrial use. An olefin sulfonate, particularly, an internal olefin sulfonate obtained with an internal olefin having a double bond inside an olefin chain, not at its end, as a raw material, has been reported as one of the anionic surfactants.

Such an internal olefin sulfonate is generally obtained by sulfonating an internal olefin through reactions with a gaseous sulfur trioxide-containing gas, followed by neutralization and then hydrolysis of the resulting sulfonic acid. The internal olefin sulfonate is known to have good biodegradability or the like, but is still insufficient in a basic performance as cleansing agents including foamability and foam quality, compared with general-purpose surfactants such as salts of alkyl polyoxyalkylene sulfuric acid esters. Thus, further improvement in such basic performance has been desired. As more people have concerned the water-saving in recent years, the additional value of foam dissipation property in addition to good foamability, foam quality, and foaming speed has also been required for use as active ingredients in laundry detergents, dishwashing detergents, shampoos or the like.

JP-A-2003-81935 discloses a specific internal olefin sulfonic acid for the purposes of the solubilizing ability, penetrating ability, and interfacial tension reducing ability. It discloses that when it is used as a shampoo, it lathers well without friction, and achieves an improved feel. U.S. Pat. No. 5,078,916 describes a specific internal olefin sulfonate for the purposes of improving detergency, and discloses examples of application to shampoos and the like.

### SUMMARY OF THE INVENTION

The present invention provides an internal olefin sulfonate composition comprising (A) an internal olefin sulfonate having 16 carbon atoms and (B) an internal olefin sulfonate having 18 carbon atoms, wherein a mass content ratio (A/B) of component (A) to component (B) is from 75/25 to 90/10.

Also, the present invention provides a cleansing composition comprising the aforementioned internal olefin sulfonate composition.

### DETAILED DESCRIPTION OF THE INVENTION

However, further improvement is still required for any of the compositions described in the documents to exert good foamability at the same time with foam quality, foaming speed, and foam dissipation property at high levels.

Therefore, the present invention is to provide an internal olefin sulfonate composition which can exert good foamability at the same time with foam quality, foaming speed, and foam dissipation property at high levels, and to provide a cleansing composition containing the same.

The present inventor studied a length of an aliphatic chain in an internal olefin sulfonate, a ratio thereof and other various conditions, and consequently found that an internal olefin sulfonate composition which satisfies good foamability, foam quality, foaming speed, and foam dissipation property at the same time can be obtained by setting the ratio between an internal olefin sulfonate having 16 carbon atoms and an internal olefin sulfonate having 18 carbon atoms to a predetermined range. On the basis of these findings, the present invention has been completed.

According to the present invention, it can provide an internal olefin sulfonate composition which can exert good foamability at the same time with foam quality, foaming speed and foam dissipation property at high levels, and to provide a cleansing composition.

Hereinbelow, the present invention will be described in detail.

<Internal Olefin Sulfonate Composition>

The internal olefin sulfonate composition of the present invention includes (A) an internal olefin sulfonate having 16 carbon atoms and (B) an internal olefin sulfonate having 18 carbon atoms, wherein a mass content ratio (A/B) of component (A) to component (B) is from 75/25 to 90/10.

In the present invention, an internal olefin sulfonate is a sulfonate obtained by sulfonating an internal olefin (an olefin having a double bond inside the olefin chain) as the raw material, followed by neutralization and then hydrolysis, as described above. It should be noted that the above internal olefin may also include a trace amount of so-called α-olefin, in which a double bond is present at the C-1 position of the carbon chain. That is, sulfonation of an internal olefin quantitatively produces β-sultone, some of which are converted into γ-sultone and olefin sulfonic acid, which are further converted into hydroxyalkane sulfonate and olefin sulfonate in the process of neutralization and hydrolysis (for example, J. Am. Oil Chem. Soc. 69, 39 (1992)). Here, the hydroxyl group of the hydroxyalkane sulfonate thus obtained is present inside the alkane chain, and the double bond of the olefin sulfonate is present inside the olefin chain. Also, the product thus obtained is mainly a mixture of the aforementioned substances, some of which may include a trace amount of hydroxyalkane sulfonate having a hydroxyl group at the end of the carbon chain or olefin sulfonate having a double bond at the end of the carbon chain. In the present specification, each of these products and a mixture thereof are collectively referred to as internal olefin sulfonate. Hydroxyalkane sulfonate is referred to as the hydroxy form of an internal olefin sulfonate (hereinbelow, may also be referred to as HAS), and olefin sulfonate is referred to as the olefin form of an internal olefin sulfonate (hereinbelow, may also be referred to as IOS).

The mass content ratio (A/B) of component (A) to component (B) contained in the internal olefin sulfonate composition of the present invention is from 75/25 to 90/10 from the viewpoint of foamability, foam quality, foaming speed, and foam dissipation property, and is preferably from 75/25 to 85/15, more preferably from 77/23 to 85/15, and even more preferably from 78/22 to 85/15 from the viewpoint of foaming speed and foam dissipation property. Also, the mass content ratio is more preferably from 80/20 to 90/10 and even more preferably from 85/25 to 90/10 from the viewpoint of foamability and the volume of foam. Further, the mass content ratio is preferably from 75/25 to 85/15 and more preferably from 75/25 to 80/20 from the viewpoint of foam quality.

The mass content ratio (A/B) of component (A) to component (B) in the internal olefin sulfonate composition may be measured by a high-performance liquid chromatograph-mass spectrometer (hereinbelow, abbreviated as HPLC-MS). Specifically, an internal olefin sulfonate having 16 carbon atoms and an internal olefin sulfonate having 18 carbon atoms are separated by HPLC, each of which may then be identified by analysis with MS. From the HPLC-MS peak area thereof, the mass content ratio (A/B) of component (A) to component (B) in the internal olefin sulfonate may be obtained.

The total content of component (A) and component (B) in the internal olefin sulfonate composition of the present invention is preferably 50% by mass or more, more preferably 70% by mass or more, more preferably 80% by mass or more, more preferably 90% by mass or more, more preferably 95% by mass or more, more preferably 96.5% by mass or more, and even more preferably 97% by mass or more from the viewpoint of foamability and foam quality. The upper limit of the total content of component (A) and component (B) is preferably 100% by mass.

As is apparent from the aforementioned production method, the sulfonate group in the internal olefin sulfonate of the present invention is present inside the olefin chain or alkane chain. In the present invention, from the viewpoint of foamability, it is preferable that the content of an internal olefin sulfonate in which the sulfonate group is present at the C-2 position of the olefin chain or alkane chain is low, while the content of an internal olefin sulfonate in which the sulfonate group is present further inside is high. It is more preferable that the content of an internal olefin sulfonate in which the sulfonate group is present at the C-2 position of the olefin chain or alkane chain is low, with respect to both of the above internal olefin sulfonates having 16 carbon atoms and 18 carbon atoms.

The content of the internal olefin sulfonate in which a sulfonate group is present at a C-2 position in the internal olefin sulfonate having 16 and 18 carbon atoms of the present invention is preferably less than 20% by mass and more preferably less than 18% by mass from the viewpoint of foamability. Also, the content is preferably 5% by mass or more and more preferably 7% by mass or more from the viewpoint of cost and productivity. Further, the content of the internal olefin sulfonate in which a sulfonate group is present at a C-2 position in the internal olefin sulfonate having 16 and 18 carbon atoms is preferably 5% by mass or more and less than 20% by mass, more preferably 7% by mass or more and less than 20% by mass, and even more preferably 7% by mass or more and less than 18% by mass from the viewpoint of foaming speed and foam dissipation property. Also, the content of the α-olefin sulfonate in which the sulfonate group is positioned at the C-1 position of an olefin chain or an alkane chain is preferably less than 2.8% by mass, more preferably 0.01% by mass or more and less than 2.8% by mass, more preferably 0.1% by mass or more and less than 2.8% by mass, and even more preferably 0.3% by mass or more and less than 2.8% by mass from the viewpoint of foamability and foam dissipation property. Here, the content of the internal olefin sulfonate having 16 and 18 carbon atoms in which the sulfonate group is present at the C-2 position is roughly consistent with the content of the raw material internal olefin in which the double bond is present at the C-2 position.

The content of the internal olefin sulfonate in which the sulfonate group is present at the C-2 position in the internal olefin sulfonate having 16 and 18 carbon atoms (A) may be measured by a method such as nuclear magnetic resonance spectroscopy.

The mass content ratio (hydroxy form/olefin form) of the hydroxy form to the olefin form in the internal olefin sulfonate having 16 and 18 carbon atoms is preferably from 50/50 to 100/0, more preferably 60/40 to 100/0, more preferably from 70/30 to 100/0, more preferably from 75/25 to 100/0, and even more preferably from 75/25 to 95/5 from the viewpoint of foamability.

The mass content ratio of the hydroxy form to the olefin form in the internal olefin sulfonate having 16 and 18 carbon atoms of the present invention may be measured by the method described later in Examples.

As the internal olefin sulfonate composition of the present invention is obtained by sulfonating an internal olefin, followed by neutralization and hydrolysis as described above, an unreacted raw material internal olefin and inorganic compounds may remain in the composition. It is preferred that the contents of these components are much smaller.

The content of the raw material internal olefin in the internal olefin sulfonate composition of the present invention is preferably less than 5.0% by mass, more preferably less than 3.0% by mass, more preferably less than 1.5% by mass, and even more preferably less than 1.0% by mass with respect to the amount of the internal olefin sulfonates from the viewpoint of foamability.

The content of the unreacted internal olefin may be measured by a method described later in Examples.

The content of the inorganic compounds in the internal olefin sulfonate composition of the present invention is preferably less than 7.5% by mass, more preferably less than 5.0% by mass, and even more preferably less than 3.0% by mass with respect to the amount of the internal olefin sulfonates from the viewpoint of foamability and foam quality.

In this context, the inorganic compounds include sulfates and alkali agents. The content of these inorganic compounds may be measured by a potentiometric titration. Specifically, the content may be measured by a method described later in Examples.

The internal olefin sulfonate composition of the present invention may contain a hydroxy form and an olefin form having any number of carbon atoms which are different from that of component (A) and component (B). The numbers of carbon atoms in the hydroxy form and the olefin form are preferably from 8 to 24, more preferably from 12 to 20, more preferably from 12 to 18, more preferably from 14 to 18, and even more preferably from 16 to 18 from the viewpoint of foamability, foaming speed, and foam dissipation property. These hydroxy forms and olefin forms having various numbers of carbon atoms are derived from the internal olefin used as a raw material.

The internal olefin sulfonate composition of the present invention may contain other components, for example, water as a medium, a pH adjuster, a viscosity reducing agent, an organic solvent, and polyhydric alcohols, in addition to the components described above.

### <Method for Producing Internal Olefin Sulfonate Composition>

The internal olefin sulfonate composition may be produced by sulfonating an internal olefin having 8 to 24 carbon atoms, followed by neutralization and hydrolysis. More specifically, for example, the composition may be produced in accordance with the methods described in U.S. Pat. Nos. 1,633,184 and 2,625,150, and Tenside Surf. Det. 31 (5) 299 (1994), and the like.

As mentioned above, in the present invention, a internal olefin refers to an olefin substantially having a double bond inside the olefin chain. The content of the α-olefin in which a double bond is present at a C-1 position is preferably less than 2.8% by mass, more preferably 0.01% by mass or more and less than 2.8% by mass, more preferably 0.1% by mass or more and less than 2.8% by mass, and even more preferably 0.3% by mass or more and less than 2.8% by mass from the viewpoint of foamability and foam dissipation property. From the viewpoint of the foamability, foaming speed, and foam dissipation property of the internal olefin sulfonate composition obtained thus, the number of carbon atoms in the internal olefin is preferably from 8 to 24, more preferably from 12 to 20, more preferably from 12 to 18, more preferably from 14 to 18, and even more preferably from 16 to 18. An internal olefin to be used may be used singly, or a combination of two or more thereof may be used.

When the internal olefin sulfonate composition is obtained by sulfonating the internal olefin, followed by neutralization and hydrolysis, the content of an internal olefin in which the double bond is present at the C-2 position in the raw material internal olefin is more preferably less than 20% by mass, and even more preferably less than 18% by mass. Also, the lower limit thereof is preferably 5% by mass or more, and more preferably 7% by mass or more.

In the synthesis of the internal olefin sulfonate composition, the content of the internal olefin in which the double bond is present at the C-2 position in the raw material internal olefin may be measured by, for example, a gas chromatograph mass spectrometer (hereinbelow, abbreviated as GC-MS). Specifically, components each having different carbon chain lengths and double bond positions are accurately separated by a gas chromatograph analyzer (hereinbelow, abbreviated as GC), and each component is then analyzed by a mass spectrometer (hereinbelow, abbreviated as MS) to identify the position of double bond. From the resulting GC peak area, the fraction of each component can be found out.

The internal olefin may contain a paraffin component. The content of the paraffin component is preferably less than 5% by mass and more preferably less than 3% by mass from the viewpoint of foamability.

The content of the paraffin component may be measured by, for example, GC-MS.

The sulfonation reaction may be carried out by reacting a sulfur trioxide gas with an internal olefin at a ratio of from 1 to 1.2 moles of sulfur trioxide per mole of the internal olefin. The reactions may be carried out at a reaction temperature of from 20 to 40° C.

Neutralization is carried out by reacting from 1 to 1.5 times the molar amount of an alkaline aqueous solution such as sodium hydroxide, potassium hydroxide, ammonia or 2-aminoethanol with the theoretical value of sulfonate group.

The hydrolysis reaction may be carried out at from 90 to 200° C. for from 30 minutes to three hours in the presence of water. These reactions may be successively carried out. Also, upon completion of the reactions, the products may be purified by extraction, washing, or the like.

Also, in the production of the internal olefin sulfonate composition, the raw material internal olefin in which the number of carbon atoms is distributed in from 8 to 24 may be subjected to sulfonation, neutralization, and hydrolysis, or the raw material internal olefin having a uniform number of carbon atoms may be subjected to sulfonation, neutralization, and hydrolysis. Also, a plurality of internal olefin sulfonates each having different numbers of carbon atoms may be produced in advance and then mixed, as needed.

The internal olefin sulfonate composition of the present invention exerts good foamability at the same time with foam quality, foaming speed, and foam dissipation property at high levels, and is thus useful as a cleansing ingredient. Specifically, the internal olefin sulfonate composition of the present invention can be used in household cleansing agents such as hair shampoos, body cleansers, laundry detergents, and kitchen detergents, and is particularly useful as a base for the hair shampoo.

### <Cleansing Composition>

The cleansing composition of the present invention is not particularly limited as long as the cleansing composition contains the internal olefin sulfonate composition of the present invention. The cleansing composition of the present invention may contain other components depending on the intended purpose. Examples of the other components include other surfactant, a foaming promoting agent, and an auxiliary agent. The content of the internal olefin sulfonate composition in the cleansing composition is preferably from 0.1 to 80% by mass, more preferably from 1 to 50% by mass, and even more preferably from 2 to 30% by mass, in terms of the amount of the internal olefin sulfonates.

The other surfactant is preferably, for example, alkyl sulfate and alkyl polyoxyalkylene sulfate. Examples of the auxiliary agent include; but not particularly limited to, water, polymer, an oil solution, silicone, a moisturizing agent, a viscosity regulator, a preservative, an anti-inflammatory agent, an antioxidant, an ultraviolet absorber, a sequestering agent, a pearlescent agent, a dye, a fragrance, an enzyme, a bleaching agent, a bleach activator, and pH adjuster.

The cleansing composition of the present invention may be produced, for example, by mixing the internal olefin sulfonate composition and the components described above.

Hereinafter, the present invention and preferable embodiments of the present invention will be described.

<1> An internal olefin sulfonate composition comprising (A) an internal olefin sulfonate having 16 carbon atoms and (B) an internal olefin sulfonate having 18 carbon atoms, wherein a mass content ratio (A/B) of component (A) to component (B) is from 75/25 to 90/10.
<2> The internal olefin sulfonate composition according to <1>, wherein the mass content ratio (A/B) of component (A) to component (B) in the internal olefin sulfonate composition is preferably from 77/23 to 85/15, and more preferably from 78/22 to 85/15.
<3> The internal olefin sulfonate composition according to <1> or <2>, wherein a total content of component (A) and component (B) in the internal olefin sulfonate composition is preferably 50% by mass or more, more preferably 70% by mass or more, more preferably 80% by mass or more, more preferably 90% by mass or more, more preferably 95% by mass or more, more preferably 96.5% by mass or more, and even more preferably 97% by mass or more, with its upper limit being 100% by mass.
<4> The internal olefin sulfonate composition according to any of <1> to <3>, wherein a content of an internal olefin sulfonate in which a sulfonate group is present at a C-2 position in the internal olefin sulfonate having 16 and 18 carbon atoms is preferably less than 20% by mass, more preferably less than 18% by mass and is preferably 5% by mass or more and more preferably 7% by mass or more.
<5> The internal olefin sulfonate composition according to any of <1> to <4>, wherein a mass content ratio of a hydroxy form to an olefin form (hydroxy form/olefin form) in the internal olefin sulfonate having 16 and 18 carbon atoms is preferably from 50/50 to 100/0, more preferably from 60/40 to 100/0, more preferably from 70/30 to 100/0, more preferably from 75/25 to 100/0, and even more preferably from 75/25 to 95/5.
<6> The internal olefin sulfonate composition according to any of <1> to <5>, wherein a content of a raw material internal olefin in the internal olefin sulfonate composition is preferably less than 5.0% by mass, more preferably less than 3.0% by mass, more preferably less than 1.5% by mass, and even more preferably less than 1.0% by mass with respect to the amount of the internal olefin sulfonates.
<7> The internal olefin sulfonate composition according to any of <1> to <6>, wherein a content of inorganic compounds in the internal olefin sulfonate composition is preferably less than 7.5% by mass, more preferably less than 5.0% by mass, and even more preferably less than 3.0% by mass with respect to the amount of the internal olefin sulfonates.
<8> The internal olefin sulfonate composition according to any of <1> to <7>, wherein the numbers of carbon atoms in a hydroxy form and an olefin form having carbon atoms other than component (A) and component (B) in the internal olefin sulfonate composition is preferably from 8 to 24, more preferably from 12 to 20, more preferably from 12 to 18, more preferably from 14 to 18, and even more preferably 16 to 18.
<9> The internal olefin sulfonate composition according to any of <1> to <8>, obtained by preferably sulfonating an internal olefin composition containing an internal olefin, followed by neutralization and then hydrolysis, wherein a content of the internal olefin in which a double bond is present at a C-2 position is less than 20% by mass.
<10> A cleansing composition comprising the internal olefin sulfonate composition according to any of <1> to <9>.
<11> The cleansing composition according to <10>, wherein a content of the internal olefin sulfonate composition is from 0.1 to 80% by mass.
<12> The cleansing composition according to <10> or <11>, further comprising one or more preferably selected from an alkyl sulfate and an alkyl polyoxyalkylene sulfate.

### EXAMPLES

Hereinbelow, the present invention will be specifically described with reference to Examples. It should be noted that unless otherwise specifically noted, the content of each of the components is expressed by % by mass in the following Tables. Also, the methods for measuring various physical properties are as follows.

### (1) Conditions of Measurement

### (i) Method for Measuring the Position of a Double Bond in the Internal Olefin

The position of a double bond in an internal olefin was measured by gas chromatography (hereinbelow, abbreviated as GC). Specifically, an internal olefin was converted to a dithiated derivative by reaction with dimethyl disulfide, and then each component was separated by GC. The position of a double bond in an internal olefin was found based on the peak area of each component.

The apparatus and analytical conditions used for the measurement are as follows. GC apparatus (trade name: HP6890, the product of Hewlett-Packard Company); Column (trade name: Ultra-Alloy-1HT capillary column, 30 m×250 μm×0.15 μm, the product of Frontier Laboratories Ltd.); Detector (hydrogen flame ionization detector (FID)); Injection temperature of 300° C.; Detector temperature of 350° C.; and He flow rate of 4.6 mL/min.

### (ii) Method for Measuring the Mass Ratio of Hydroxy Form/Olefin Form

The mass ratio of hydroxy form/olefin form was measured by HPLC-MS. Specifically, the hydroxy form and the olefin form were separated by HPLC and each form was identified by separately analyzing with MS. From the resulting GC-MS peak area, the fraction of each form was obtained.

The apparatus and analytical conditions used for the measurement are as follows. HPLC apparatus (trade name: Agilent technology 1100, the product of Agilent Technologies, Inc.); Column (trade name: L-column ODS 4.6×150 mm, the product of Chemicals Evaluation and Research Institute, Japan); Sample preparation (diluted 1000-fold with methanol); Eluent A (10 mM ammonium acetate in water); Eluent B (10 mM ammonium acetate in methanol), Gradient (0 min (A/B=30/70%)→10 min (30/70%)→55 min (0/100%)→65 min (0/100%)→66 min (30/70%)→75 min (30/70%); MS apparatus (trade name: Agilent technology 1100 MS SL (G1946D); and MS detection (anion detection m/z 60-1600, UV 240 nm).

(iii) Method for Measuring the Content of the Raw Material Internal Olefin

The content of the raw material internal olefin was measured by GC. Specifically, ethanol and petroleum ether were added to an aqueous solution of internal olefin sulfonate, followed by extraction to give olefin in the petroleum ether phase. From the GC peak area of the olefin, the amount thereof was quantitated.

The apparatus and analytical conditions used for the measurement are as follows. GC apparatus (trade name: Agilent technology 6850, the product of Agilent Technologies, Inc.); Column (trade name: Ultra-Alloy-1HT capillary column, 15 m×250 μm×0.15 μm, the product of Frontier Laboratories, Ltd.); Detector (hydrogen flame ionization detector (FID)); Injection temperature of 300° C.; Detector temperature of 350° C.; and He flow rate of 3.8 mL/min.

### (iv) Method for Measuring the Content of Inorganic Compounds

The content of inorganic compounds was measured by potentiometric titration and neutralization titration. Specifically, the content of Na₂SO₄ was quantitated by measuring sulfate ion (SO₄²⁻) by potentiometric titration. Also, the content of NaOH was quantitated by neutralization titration with diluted hydrochloric acid.

### (v) Method for Measuring the Content of the Paraffin Component

The content of the paraffin component was measured by GC. Specifically, ethanol and petroleum ether were added to an aqueous solution of internal olefin sulfonate, followed by extraction to give paraffin in the petroleum ether phase. From the GC peak area of the paraffin, the amount thereof was quantitated.

It should be noted that the apparatus and analytical conditions used are the same as those used for the measurement of the content of the raw material internal olefin.

### (2) Production of an Internal Olefin

### Production Example A

### Synthesis of C16 Internal Olefins in which 16.5% by Mass of Double Bonds was Present at C-2 Position

Into a flask with a stirrer, 7000 g (28.9 moles) of 1-hexadecanol (trade name: KALCOL 6098, the product of Kao Corporation), and as a solid acid catalyst, 700 g (10% by mass relative to the raw material alcohol) of γ-alumina (STREM Chemicals, Inc.) were placed, and reactions were allowed to proceed for five hours at 280° C. while stirring and passing nitrogen (7000 mL/minute) through the system. The alcohol conversion ratio was 100% and the purity of C16 internal olefin was 99.7% after the completion of the reaction. The resulting crude internal olefin was transferred to a distillation flask and distilled at from 136 to 160° C./4.0 mmHg, whereby 100% pure internal olefin having 16 carbon atoms was obtained. The double bond distribution in the resulting internal olefin was as follows: C-1 position, 0.5% by mass; C-2 position, 16.5% by mass; C-3 position, 15.4% by mass; C-4 position, 16.4% by mass; C-5 position, 17.2% by mass; C-6 position, 14.2% by mass; and C-7 and 8 positions, 19.8% by mass in total.

### Production Example B

### Synthesis of C18 Internal Olefins in which 16.9% by Mass of Double Bonds was Present at C-2 Position

Into a flask with a stirrer, 7000 g (25.9 moles) of 1-octadecanol (trade name: KALCOL 8098, the product of Kao Corporation), and as a solid acid catalyst, 1050 g (15 wt % relative to the raw material alcohol) of γ-alumina (STREM Chemicals, Inc.) were placed, and reactions were allowed to proceed for 13 hours at 285° C. while stirring and passing nitrogen (7000 mL/minute) through the system. The alcohol conversion ratio was 100% and the purity of C18 internal olefin was 98.5% after the completion of the reaction. The resulting crude internal olefin was transferred to a distillation flask and distilled at from 148 to 158° C./0.5 mmHg, whereby 100% pure internal olefin having 18 carbon atoms was obtained. The double bond distribution in the resulting internal olefin was as follows: C-1 position, 0.7% by mass; C-2 position, 16.9% by mass; C-3 position, 15.9% by mass; C-4 position, 16.0% by mass; C-5 position, 14.7% by mass; C-6 position 11.2% by mass; C-7 position, 10.2% by mass; and C-8 and 9 positions, 14.6% by mass in total.

### Production Example C

### Synthesis of C16 Internal Olefins in which 30.4% by Mass of Double Bonds was Present at C-2 Position

Into a flask with a stirrer, 7000 g (28.9 moles) of 1-hexadecanol (trade name: KALCOL 6098, the product of Kao Corporation), and as a solid acid catalyst, 700 g (10 wt % relative to the raw material alcohol) of γ-alumina (STREM Chemicals, Inc.) were placed, and reactions were allowed to proceed for three hours at 280° C. while stirring and passing nitrogen (7000 mL/minute) through the system. The alcohol conversion ratio was 100% and the purity of C16 internal olefin was 99.6% after the completion of the reaction. The resulting crude internal olefin was transferred to a distillation flask and distilled at from 136 to 160° C./4.0 mmHg, whereby 100% pure internal olefin having 16 carbon atoms was obtained. The double bond distribution in the resulting internal olefin was as follows: C-1 position, 1.8% by mass; C-2 position, 30.4% by mass; C-3 position, 23.9% by mass; C-4 position, 16.8% by mass; C-5 position, 12.0% by mass; C-6 position, 7.4% by mass; and C-7 and 8 positions, 7.8% by mass in total.

### Production Example D

### Synthesis of C18 Internal Olefins in which 31.3% by Mass of Double Bonds was Present at C-2 Position

Into a flask with a stirrer, 7000 g (25.9 moles) of 1-octadecanol (trade name: KALCOL 8098, the product of Kao Corporation), and as a solid acid catalyst, 700 g (10% by mass relative to the raw material alcohol) of γ-alumina (STREM Chemicals, Inc.) were placed, and reactions were allowed to proceed for 10 hours at 280° C. while stirring and passing nitrogen (7000 mL/minute) through the system. The alcohol conversion ratio was 100% and the purity of C18 internal olefin was 98.2% after the completion of the reaction. The resulting crude internal olefin was transferred to a distillation flask and distilled at from 148 to 158° C./0.5 mmHg, whereby 100% pure purified internal olefin was obtained. The double bond distribution in the resulting internal olefin was as follows: C-1 position, 0.8% by mass; C-2 position, 31.3% by mass; C-3 position, 22.9% by mass; C-4 position, 15.5% by mass; C-5 position, 10.8% by mass; C-6 position, 7.2% by mass; C-7 position, 5.3% by mass; and C-8 and 9 positions, 6.2% by mass in total.

### Production Example E

### Synthesis of C14 Internal Olefins in which 31.8% by Mass of Double Bonds was Present at C-2 Position

A flask with a stirrer was charged with 6000 g (26.7 moles) of 1-tetradecene (product name: Linealene 14, the product of Idemitsu Kosan Co., Ltd.) and 180 g (3% by mass relative to the amount of the raw material α-olefin) of protonic β-zeolite (CP-814E, Zeolyst Int.) as a solid acid catalyst, followed by reaction at 120° C. for 20 hours with stirring. Subsequently, the crude internal olefins were transferred to a flask for distillation and distilled at from 124-136° C./7.5 mmHg, to obtain C14 internal olefins having olefin purity of 100%. The double bond distribution of the resulting internal olefins was 1.3% by mass at a C-1 position, 31.8% by mass at a C-2 position, 23.8% by mass at a C-3 position, 21.0% by mass at a C-4 position, 8.6% by mass at a C-5 position, and 13.6% by mass in total at C-6 and C-7 positions.

### (2) Production of an Internal Olefin Sulfonate

### Production Example 1

Using a thin film sulfonation reactor having an outer jacket, the sulfonation reaction of the internal olefin having 16 carbon atoms (the content of an internal olefin in which a double bond is present at a C-2 position is 16.5% by mass) obtained in Production Example A was carried out by passing through sulfur trioxide gas, while passing cooling water of 20° C. through the outer jacket of the reactor. The molar ratio of SO₃/internal olefin for the sulfonation reaction was set at 1.09. The resulting sulfonation product was added to an alkaline aqueous solution prepared with 1.5 times the molar amount of sodium hydroxide relative to the theoretical acid value, followed by neutralization at 30° C. for one hour while stirring. The resulting neutralized product was hydrolyzed by heating at 160° C. for one hour in an autoclave, whereby a crude product of sodium C16 internal olefin sulfonate was obtained. Then, 300 g of the crude product was transferred to a separatory funnel, to which 300 mL of ethanol was added and then 300 mL of petroleum ether was added per operation, whereby oil-soluble impurities were removed by extraction. At this time, inorganic compounds (mainly composed of sodium sulfate) which were precipitated at the oil-water interface by the addition of ethanol were also separated and removed from the aqueous phase by the oil-water separation operation. The above removal/extraction operation was repeated three times. Then, the aqueous phase side was evaporated to dryness, whereby sodium C16 internal olefin sulfonate was obtained. The mass ratio of hydroxy form (sodium hydroxyalkane sulfonate)/olefin form (sodium olefin sulfonate) in the obtained sodium internal olefin sulfonate was 81/19. Also, the content of the raw material internal olefin contained in the obtained sodium internal olefin sulfonate was less than 100 ppm (less than GC detection limits), while the content of inorganic compounds therein was 1.3% by mass.

### Production Example 2

A sodium C18 internal olefin sulfonate was obtained under the same conditions as those used in Production Example 1 from the internal olefin having 18 carbon atoms (the content of an internal olefin in which a double bond is present at a C-2 position is 16.9% by mass) obtained in Production Example B.

The mass ratio of hydroxy form/olefin form in the obtained sodium internal olefin sulfonate was 80/20. Also, the content of the raw material internal olefin contained in the obtained sodium internal olefin sulfonate was less than 100 ppm (below the GC detection limit) and that of inorganic compounds was 1.7% by mass.

### Production Example 3

A sodium C16 internal olefin sulfonate was obtained under the same conditions as those used in Production Example 1 from the internal olefin having 16 carbon atoms (the content of an internal olefin in which a double bond is present at a C-2 position is 30.4% by mass) obtained in Production Example C.

The mass ratio of hydroxy form/olefin form in the obtained sodium internal olefin sulfonate was 90/10. Also, the content of the raw material internal olefin contained in the obtained sodium internal olefin sulfonate was less than 100 ppm (below the GC detection limit) and that of inorganic compounds was 1.9% by mass.

### Production Example 4

A sodium C18 internal olefin sulfonate was obtained under the same conditions as those used in Production Example 1 from the internal olefin having 18 carbon atoms (the content of an internal olefin in which a double bond is present at a C-2 position is 31.3% by mass) obtained in Production Example D.

The mass ratio of hydroxy form/olefin form in the obtained sodium internal olefin sulfonate was 80/20. Also, the content of the raw material internal olefin contained in the obtained sodium internal olefin sulfonate was less than 100 ppm (below the GC detection limit) and that of inorganic compounds was 0.9% by mass.

### Production Example 5

Using a thin film sulfonation reactor having an external jacket, the sulfonation reaction of the internal olefins having 18 carbon atoms (the content of an internal olefin in which a double bond was present at a C-2 position was 16.9% by mass) obtained in Production Example B was carried out by passing through sulfur trioxide gas, while passing cooling water of 20° C. through the outer jacket of the reactor. The molar ratio of SO₃/internal olefin for the sulfonation reaction was set at 1.09. The resulting sulfonation product was transferred to a round-bottom flask and aged by heating at 40° C. for 30 minutes while stirring. Subsequently, the resulting product was added to an aqueous alkali solution prepared with 1.5 times the molar amount of sodium hydroxide relative to the theoretical acid value, followed by neutralization at 30° C. for one hour while stirring. The resulting neutralized product was hydrolyzed by heating at 160° C. for one hour in an autoclave, whereby a crude product of sodium C18 internal olefin sulfonate was obtained. Then, 300 g of the crude product was transferred to a separatory funnel, to which 300 mL of ethanol was added and then 300 mL of petroleum ether was added per operation. The extraction operation was carried out three times. The aqueous phase was evaporated to dryness to obtain a sodium C18 internal olefin sulfonate. The mass ratio of hydroxy form (sodium hydroxyalkane sulfonate)/olefin form (sodium olefin sulfonate) in the obtained sodium internal olefin sulfonate was 57/43. Also, the content of the raw material internal olefin contained in the obtained sodium internal olefin sulfonate was 0% by mass, while the content of inorganic compounds therein was 1.2% by mass.

### Production Example 6

### Synthesis of C14 Internal Olefin Sulfonate

A sodium C14 internal olefin sulfonate was obtained under the same conditions as in Production Example 1 from the internal olefin having 14 carbon atoms (the content of an internal olefin in which a double bond was present at a C-2 position was 31.8% by mass) obtained in Production Example E.

The mass ratio of hydroxy form/olefin form in the obtained sodium internal olefin sulfonate was 93/7. Also, the content of the raw material internal olefin contained in the obtained sodium internal olefin sulfonate was 0% by mass and that of inorganic compounds therein was 0% by mass.

### Production Example 7

The composition obtained in Production Example 1 and the composition obtained in Production Example 2 were mixed at a mass ratio of 80:20 to obtain internal olefin sulfonate composition 1.

### Production Example 8

The composition obtained in Production Example 1 and the composition obtained in Production Example 2 were mixed at a mass ratio of 90:10 to obtain internal olefin sulfonate composition 2.

### Production Example 9

The composition obtained in Production Example 1 and the composition obtained in Production Example 2 were mixed at a mass ratio of 75:25 to obtain internal olefin sulfonate composition 3.

### Production Example 10

The composition obtained in Production Example 6, the composition obtained in Production Example 1, and the composition obtained in Production Example 2 were mixed at a mass ratio of 50:40:10 to obtain internal olefin sulfonate composition 4.

### Production Example 11

The composition obtained in Production Example 1 and the composition obtained in Production Example 5 were mixed at a mass ratio of 75:25 to obtain internal olefin sulfonate composition 5.

### Production Example 12

The composition obtained in Production Example 3 and the composition obtained in Production Example 4 were mixed at a mass ratio of 80:20 to obtain internal olefin sulfonate composition 6.

### <Hair Evaluation>

A hair bundle (hair of a Japanese person free from treatment such as bleach or hair color; approximately 20 cm, 15 g) was cleansed with a plain shampoo shown below. Then, after application of a plain rinse shown in the table below, the hair bundle was rinsed off with tap water to obtain a tress for evaluation.

Each of the compositions obtained in Production Examples 7 to 10 was dissolved in ion-exchange water to prepare an aqueous solution (13% by mass) of the internal olefin sulfonate composition. Using these aqueous solutions, five expert panelists evaluated their foamability, foam quality, foaming speeds, and foam dissipation in accordance with evaluation criteria and evaluation methods shown below (specifically, 1.0 g of each cleansing composition shown in Table 3 was applied to the tress for evaluation and subjected to lathering, cleansing, and then rinsing). The results are shown in Table 3. Table 3 also shows results of evaluating alkyl polyoxyethylene sulfate (AES), α-olefin sulfonate (AOS), and secondary alkyl sulfonate (SAS).

TABLE 1
(Composition of plain shampoo)

| (Component) | (%) |
|---|---|
| Sodium polyoxyethylene lauryl ether sulfate | 11.3 |
| (42.0% in terms of EMAL E-27C (manufactured by Kao Corp.; | |
| 27% by weight of active component)) | |
| Coconut oil fatty acid N-methylethanolamide | 3.0 |
| (AMINON C-11S (manufactured by Kao Corp.)) | |
| Citric acid | 0.2 |
| Methylparaben | 0.3 |
| Purified water | Balance |
| Total | 100.0 |

### (Production of Plain Shampoo)

The components were placed in a beaker, heated to 80° C., and then mixed. After confirmation of uniform dissolution, the mixture was cooled to obtain a plain shampoo.

TABLE 2
(Composition of plain rinse)

| (Component) | (%) |
|---|---|
| Octadecyloxypropyl trimethyl ammonium chloride | 3.0 |
| (6.7% in terms of QUARTAMIN E-80K (manufactured by Kao | |
| Corp.; 45% by weight of active component)) | |
| Stearyl alcohol | 6.0 |
| (KALCOL 8098 (manufactured by Kao Corp.)) | |
| Methylparaben | 0.3 |
| Purified water | Balance |
| Total | 100.0 |

### (Production of Plain Rinse)

Octadecyloxypropyl trimethyl ammonium chloride and stearyl alcohol were placed in a beaker (A) and melted by heating to 80° C. Purified water and methylparaben were placed in another beaker (B) and heated to 80° C. with stirring. After confirmation of uniform dissolution, the mixed solution in the beaker (A) was added to the beaker (B) with stirring at 80° C. and emulsified for 30 minutes. The heating was terminated, and it was cooled to room temperature to obtain a plain rinse.

### <Evaluation Criteria and Evaluation Methods>

Foamability

5: Foaming properties were very good
4: Foaming properties were good
3: Ordinary foamability (equivalent to Comparative Example 1: AES)
2: Foaming properties were poor
1: Foaming properties were too poor to cleanse hair

Foaming Speed

5: Lathering was very quick and facilitated cleansing
4: Lathering was quick
3: Ordinary (equivalent to Comparative Example 1: AES)
2: Lathering was slow
1: Lathering was very slow

Foam Quality

5: Foam quality was creamy and very good
4: Foam quality was slightly creamy and good
3: Foam quality was ordinary (equivalent to Comparative Example 1: AES)
2: Foam quality was slightly bubbly and poor
1: Foam quality was bubbly and very poor and hindered cleansing

Foam Dissipation

5: Foam was very quickly dissipated and easily rinsed
4: Foam was quickly dissipated
3: Ordinary (equivalent to Comparative Example 1: AES)
2: Foam was slowly dissipated
1: Foam was very slowly dissipated and hardly rinsed

### <Hand Wash Evaluation>

Five panelists washed their hands (specifically, 1.0 g of each cleansing composition shown in Table 3 was applied to the hands and subjected to lathering, cleansing, and rinsing) and evaluated each composition in accordance with the same criteria as in the hair evaluation. The results are shown in Table 3.

### <Test on Volume of Foam>

A tress treated in the same way as in the hair evaluation was used. Foam obtained by lathering in the same way as above was placed in a graduated cylinder of 5 cm in diameter made of glass, and the volume of the foam was measured. This operation was repeated three times, and an average thereof (rounded off to the closest whole number) was defined as the volume (mL) of foam.

TABLE 3

| | | | | | | | Reference Comparative | | |
|---|---|---|---|---|---|---|---|---|---|
| | | | Internal olefin sulfonate composition | | | | Examples | | |
| | | | 1 | 2 | 3 | 4 | 1 | 2 | 3 |
| Structure | Composition of alkyl | | C16/18 = | C16/18 = | C16/18 = | C14/16/18 = | AES*4 | AOS*5 | SAS*6 |
| | | | 80/20 | 90/10 | 75/25 | 50/40/10 | | | |
| | Content of C16/18 | | 100 | 100 | 100 | 50 | | | |
| | Hydroxy form/olefin form | | 80/20 | 80/20 | 80/20 | 80/20 | | | |
| | Ratio of double bond present at C-2 | | 16.6 | 16.5 | 16.6 | 16.6 | | | |
| | position in raw material internal olefin | | | | | | | | |
| Composition | Amount of internal olefin | | <100 ppm | <100 ppm | <100 ppm | <100 ppm | | | |
| | Amount (%) of inorganic compound | | 1.4 | 1.3 | 1.4 | 1.0 | | | |
| Evaluation | Hair | Foamability | 4.4 | 4.8 | 4.2 | 4.6 | 3.0 | 4.0 | 4.8 |
| results | evaluation | Volume of foam | 175 | 190 | 165 | 270 | 93 | 144 | 200 |
| | | Foaming speed | 4.0 | 4.0 | 3.8 | 4.6 | 3.0 | 3.8 | 3.4 |
| | | Foam dissipation | 4.8 | 4.4 | 4.4 | 4.4 | 3.0 | 3.8 | 2.8 |
| | | Foam quality | 2.8 | 2.4 | 2.9 | 2.2 | 3.0 | 2.4 | 1.2 |
| | Hand wash | Foamability | 3.8 | 4.0 | 3.8 | 4.6 | 3.0 | 3.0 | 4.0 |
| | evaluation | Foam dissipation | 5.0 | 5.0 | 5.0 | 5.0 | 3.0 | 4.2 | 2.0 |
| | | Foam quality | 3.0 | 2.8 | 3.1 | 2.0 | 3.0 | 2.0 | 1.8 |

*4Sodium alkyl polyoxyethylene sulfate (AES), manufactured by Kao Corp., EMAL 270S (active component: 70%)
*5Sodium α-olefin sulfonate (AOS), manufactured by Lion Corp., LIPOLAN LB-440 (active component: 36%)
*6Secondary sodium alkyl sulfonate (SAS), manufactured by LANXESS K.K., Mersolat H95 (active component: 95%)

What is claimed is:

1. A internal olefin sulfonate composition comprising (A) an internal olefin sulfonate having 16 carbon atoms and (B) an internal olefin sulfonate having 18 carbon atoms, wherein a mass content ratio (A/B) of the component (A) to the component (B) contained in the internal olefin sulfonate composition is from 75/25 to 90/10.

2. The internal olefin sulfonate composition according to claim 1, wherein a total content of (A) the internal olefin sulfonate having 16 carbon atoms and (B) the internal olefin sulfonate having 18 carbon atoms in the internal olefin sulfonate is from 50 to 100% by mass.

3. The internal olefin sulfonate composition according to claim 1, wherein a content of an internal olefin sulfonate in which a sulfonic acid group is present at a C-2 position in the internal olefin sulfonate having 16 and 18 carbon atoms is less than 20% by mass.

4. The internal olefin sulfonate composition according to any of claim 1, wherein a mass ratio (hydroxy form/olefin form) of a content of a hydroxy form in the internal olefin sulfonate having 16 and 18 carbon atoms to a content of an olefin form in the internal olefin sulfonate having 16 and 18 carbon atoms is from 50/50 to 100/0.

5. The internal olefin sulfonate composition according to any one of claim 1, wherein a content of a raw material internal olefin in the internal olefin sulfonate composition is less than 1.5% by mass with respect to the amount of the internal olefin sulfonates.

6. The internal olefin sulfonate composition according to any one of claim 1, wherein a content of inorganic compounds in the internal olefin sulfonate composition is less than 7.5% by mass with respect to the amount of the internal olefin sulfonates.

7. The internal olefin sulfonate composition according to any one of claim 1, obtained by sulfonating an internal olefin composition containing an internal olefin, followed by neutralization and then hydrolysis, a content of the internal olefin in which a double bond is present at a C-2 position being less than 20% by mass.

8. A cleansing composition comprising the internal olefin sulfonate composition according to any of claim 1.

9. The cleansing composition according to claim 8, wherein a content of the internal olefin sulfonate composition is from 0.1 to 80% by mass.

10. The cleansing composition according to claim 8, further comprising one or more selected from an alkyl sulfate and an alkyl polyoxyalkylene sulfate.
